(19) United States
(12) Patent Application Publication
(10) Pub. No.: US 2021/0082557 A1
(43) Pub. Date: Mar. 18, 2021

(54) PHARMACY/HEALTHCARE INFORMATION SYSTEM CARE PROVIDER, ORDER AND SERVICE MANAGEMENT SYSTEM

(71) Applicants: Naweed Muhammad, Fremont, CA (US); Kashif Rana, Fremont, CA (US)

(72) Inventors: Naweed Muhammad, Fremont, CA (US); Kashif Rana, Fremont, CA (US)

(21) Appl. No.: 16/570,229

(22) Filed: Sep. 13, 2019

(51) Int. Cl. G16H 20/10; G16H 70/40; G06Q 20/14

(52) U.S. Cl. CPC G16H 20/10; G06Q 20/14; G16H 70/40

(57) ABSTRACT

This disclosure relates generally to a system and method for the optimized management of pharmacy workflow, specifically, workflow associated with fulfilment of prescription orders for medications and health-related products in a pharmacy environment. A computer-controlled system coordinates and controls pharmacy workflow to sequence prescriptions for fulfilment in a most efficient path thereby minimizing a cost function associated with fulfilment of the prescription order. The system coordinates and controls prescription order fulfilment from automated and non-automated storage locations and can be easily adapted to the specific layout and level of automation desired by the operator. A smartphone, a laptop computer, a portable communicating device, a cloud computing device, or a data storage device is capable of communicating with the computer. Pharmaceutical care management system-connects care providers (in a hierarchical manner based on care-decision authority level), pharmacy team and delivery teams for quick, clear, and efficient communication and feedback/follow up features. Reduces errors of, unawareness about and time & effort for seeking pharmaceutical care. In order words, it makes a care-need assessment, order placement, fulfilment and status tracking a breeze.

### PRIOR REFERENCE

This application is a Provisional application of U.S. application No. 62/729,412

### BACKGROUND

### Field of the Invention

Embodiments relate to an online, data processing systems and more particularly to data processing systems used to manage delivery of healthcare services. The present invention further relates to methods and an apparatus for developing, analyzing, investigating, supporting and advising healthcare and pharmacy-related decisions.

### Description of the Related Art

Existing systems for managing the pharmaceutical care of patients lack automation and healthcare providers work in silos and rely heavily on a telephonic discussions and exchange of information about care decisions. Such communication is inefficient, error/misunderstanding-prone and lacks traceability and proper, effective and efficient coordination of care. This also requires that the information recipient be available for taking phone calls, which, in the busy world of healthcare, is not always feasible and thus results in missed and repeated calls and delay in care, which sometimes can have serious repercussions for the patient. Moreover, this method lacks an ability to follow-up, repeat, modify or cancel an order for pharmaceutical care. To alleviate this problem, a system is needed that both maximize the efficiency of the healthcare process and provides patients with a timely, disruption-free, accurate and well-documented pharmaceutical care.

Moreover, with increasing demand, the role of pharmacists is quickly moving from that of a vendor to that of a health care provider. As a result, pharmacists are increasingly concerned about advising patients regarding the selection, use and side effects of over-the-counter and prescription drugs.

There are multiple systems that have been proposed in prior art regarding pharmacy health care information system. For instance, a method and apparatus for integrated management of pharmaceutical and healthcare services bearing U.S. Pat. No. 6,112,182A is issued to HCC Inc. The patent discusses a computer-based pharmaceutical practice management system and a healthcare management system for use by a pharmacy includes a processes by which health conditions of a patient, drugs taken the patient and other information gathered by the pharmacist with the practice management system in connection with dispensing the will automatically initiate related actions handled by processes of the healthcare management system.

Another invention on flexible computer based pharmaceutical care cognitive services management system and method bearing European patent 0,811,201B1 is issued to Glaxo Wellcome Inc. The patent is about a computerized pharmaceutical care cognitive services management system and method (PC-CSMS) (24) allows the transformation of a pharmacist from a vendor to a health care provider. The PC-CSMS (24) captures all of the value added by a pharmacist to a patient encounter and permits the association of multiple RARs (Reason, Action, Result) (34) with a single SOAP (Subjective Information, Objective Information, Assessment, Plan) (32). The PC-CSMS (24) enables the pharmacist to be compensated for each analytical or counseling session (30) and/or service provided to the user associated with a single transaction. The PC-CSMS (24) also enables efficient processing of interruptions (36) to cognitive (28) and counseling (30) sessions. Upon reception of an interrupt (36) from a person or a telephone, the PC-CSMS (24) suspends the cognitive (28) or counseling (30) session for a first patient, processes the interrupt for the second patient and then resumes processing for the first patient.

A Medication management system bearing Chinese patent 1,005,04893C is issued to Chinese inventor. The patent is about a system and method for programming the device with the correct drug administration of the drug administration. Drug medical database were used to compare drug administration delivery device input parameters and guidelines or institution's widely accepted protocol, in order to ensure delivery of the drug based on these criteria. You may also be configured medical database carrier to transmit information and other information about the patient in a drug delivery system between the control and patient specific information other communication system equipment care facility such as an infusion pump. Medical database carrier may be a smart card, PDA such Palm™Pilot, computer, pager, mobile phone, or capable of storing, processing and transmitting information to other devices. The system may use a wired or wireless connection to transfer information between components of the system.

Another patent on Pharmaceutical patient information system bearing French patent 2,003,009204A2 discloses patient information system for the provision of individual-specific information on the effects and risks of a medicament with an expert system, in which relevant data on the medicament, for example, compositions, application area, contra-indications, mode of action, risks and side-effects are stored. Said expert system is embodied such that, on a user inquiry about a particular medicament by inputting particular personal status data about the user, an individual user information reply, which matches the personal status data including the education and the knowledge state of the user—(a personalised enclosure) is generated comprising all relevant data, excluding only that data on the medicament which need not be introduced according to the special requirements of the user.

A Method for analyzing side effects and interactions of pharmaceuticals bearing US patent 2,003,0144883A1 is issued to Magnus FagerholmNiklas Kvarnstrom. The patent is about a computer program for analyzing interactions between pharmaceuticals used by a patient by analyzing the prescribed pharmaceutical with a pharmaceutical profile section showing pharmaceuticals used by the patient, a diagnose profile section showing diagnose information about the patient, and an over-sensitivity profile section showing pharmaceuticals and medical substances to which the patient is sensitive. The program automatically issues warnings if the prescribed drug is incompatible with any of the pharmaceuticals or active substances of the pharmaceuticals listed in the pharmaceutical profile or over-sensitivity profile sections. The program also warns about side effects that may interfere with patient activities such as driving, breast feeding, alcohol, etc.

A Method and apparatus for determining pharmacy order parameters based on patient context data bearing US patent 2,006,0106647A1 is issued to Epic Systems Corp. The health care information system includes an information database and a pharmacy module. The information database is adapted to store a record for a patient including patient context data. The pharmacy module is adapted to receive a pharmacy order for the patient, retrieve the patient context data from the record in the information database, and determine at least one recommended parameter for the pharmacy order based on the patient context data. A method for determining pharmacy order parameters in a health care information system includes storing a record for a patient including patient context data. A pharmacy order is received for the patient. The patient context data is retrieved from the record in the information database. At least one recommended parameter for the pharmacy order is determined based on the patient context data.

There are multiple inventions that have been proposed in prior art regarding pharmacy and health management system. However, the utility of these systems has not been seen in advance form and for specialized needs. Currently most existing and traditional pharmaceutical care to institutional patients is a complicated process that requires cohesion and coordination amongst care-providers. Currently, this process is segregated into care providers' functional and locational silos resulting in inter-functional and inter-locational lack of effective and efficient coordination and information access. This is particularly true in the context of pharmaceutical care of institutional patients, where the obvious time constraints often preclude adequate, and sometimes extremely critical, intervention and/or fulfilment by other care providers. Sometimes, it results in a care decision which is not cost-effective, clinically-suited or safe. Sometimes, resulting order for pharmaceutical care cannot be fulfilled owing to time or stock constraints.

The current invention proposes a system where a new approach, utilizing the best of breed in existing technologies, to revolutionize the digital arena, implementing the bridge that fills the gap between pharmacy, hospitals and patients is implemented.

The proposed platform seeks to significantly simplify the whole process and efficiently manages the circumstances through its swift ability to connect care providers. The invention relates to the development of an automated healthcare system that addresses the needs and desires of both healthcare providers (e.g., clinicians, pharmacists, nurses and allied health professionals) and patients. The system encompasses computer communications network-based systems, software, various input and output stations in order to treat institutional patients by the provision of an updated pharmaceutical care system. The system also aims to present multiple additional features which can provide advance level of assistance to its users.

As per applicants' knowledge, none of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is therefore the purpose of the invention to alleviate at least to some extent one or more of the aforementioned problems of the prior art and/or to provide the relevant public with a suitable alternative thereto having relative advantages.

The primary object of the invention is related to the provision of an automated healthcare system that addresses the needs and desires of both healthcare providers (e.g., clinicians, pharmacists, nurses and allied health professionals) and patients. The system encompasses computer communications network-based systems, software, various input and output stations in order to treat institutional patients by the provision of an updated pharmaceutical care system.

It is also the objective of the invention to provide a simplified process of health care information system including an information database and a pharmacy module. The information database is adapted to store a record for a patient including patient context data. The pharmacy module is adapted to receive a pharmacy order for the patient, retrieve the patient context data from the record in the information database, and determine at least one recommended parameter for the pharmacy order based on the patient context data.

It is moreover the objective of the invention to provide a system which provides a method for determining pharmacy order parameters in a health care information system. The method includes storing a record for a patient including patient context data. A pharmacy order is received for the patient. The patient context data is retrieved from the record in the information database. At least one recommended parameter for the pharmacy order is determined based on the patient context data.

It is also the objective of the invention to provide a system which has provision of choosing delivery option be configured for each requesting party. This can be set for specific time or date. Furthermore, options of courier services and other indicators can also be selected.

It is moreover the objective of the invention to provide a platform, where information provided by a patient concerning the patient's lifestyle and health history may be used in combination with information about the patient gathered or recorded in connection with dispensing pharmaceutical drugs in order to initiate a healthcare related service appropriate for the patient.

It is also the objective of the invention to provide a system where when creating a new account, the new member can send the invitation to new user (i.e. new hospice, new driver and new nurses). Upon signup through that invitation, an email or text will be sent to hospice, drivers and nurses with the link of portal and/or app with login info.

It is further the objective of the invention to provide a dual and reliable system which is easy to install and is easily compatible for all types of smart devices.

It is moreover the objective of the invention to provide a system which is inline with latest technologies and utilizes the latest health care information system including but not limited to real time environment, a highly compatible and artificial intelligence-based system is introduced.

It is also the objective of the invention to provide a system which can add multiple pharmacy location and can link an account with more than one pharmacy. Each patient assigned to one or more pharmacies where one pharmacy can be selected based on patient's location and other is for back-up and/or off-hour pharmacy.

It is also the objective of the invention to provide a provision of patient's pharmacy information switched, and all related orders placed/delivered/pending (with an option to select date range or 30 days by default) are automatically (or manually) complied into a report (Recent Rx Activity) and provider can send to new pharmacy.

It is moreover the objective of the invention to provide a real time assistance for each hospice where a formulary i.e. lists of medicine which will be covered. Anything not in the formulary will be marked as non-formulary. At the time of ordering nurse will know if the medicine is formulary or non-formulary.

It is further the objective of the invention to provide an interface which is easy to use, and where new users are provided with tutorials and necessary guidance to use the system.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The current invention in its preferred embodiment discloses a new approach, linking pharmacy, hospitals and patients.

The current invention as per its preferred embodiments provides a system which has a main focus of dissemination of patient health-related decisions, assessment and timing, which is a complex process, particularly when viewed in the context of the fact that several care providers are involved—each at different expertise/responsibility level and at a different location and work schedule. Quick, accurate, retrievable and fully documented information and its flow are critical for the best outcome of care and maximum safety of the patient. This scenario becomes excessively intricate when personnel, resources are scarce and time pressure is ever mounting. Under those conditions, there is need to develop a system which allows for seamless, efficient, and accurate information management which is readily retrievable and is amenable to intervention, modification and reassessment up and down the hierarchy of providers. Recent advances in the communication industry, particularly immense computing power of handheld devices have offered an ample playing field to fabricate systems for specific and varied purposes. The current invention takes into account all those aspects and addresses a critical need for desired and essential collaboration in pharmaceutical care.

In embodiments, methods and systems for automatically maintaining an enhanced pharmaceutical care management system for a patient in accordance with various embodiments of the present invention may be provided. In embodiments, methods and systems may include an automatic data collection facility which refers to a server-based system compromising of a database compromising all automated data forms, reports, statistic and a real-time data integration facility. In embodiments, the automatic data collection facility may collect data of a medically-related event in proximity to a patient upon the occurrence of the event. The data of the medically-related event may include medication prescription, administration data that may include patient identification data that may be collected from a medication container contemporaneously with the medically-related event. In embodiments, the real-time data integration facility may be in electronic communication with the automatic data collection facility. The real-time data integration facility may automatically integrate the data of a medically-related event with a patient's EHR to maintain the enhanced EHR.

The pharmaceutical care management system has some features, which are further elaborated and defined for reference. In this embodiment, the term ‘Pharmaceutical care’ in this application refers to any and all services, whether explicitly mentioned or not, provided by a pharmacy and pharmacist. For a way of example, not limitation, it includes prescription filling, compounding, counselling a health care provider or a patient, performing screening and diagnostic test at a point-of-care, prescription suitability and safety evaluation, and prescription delivery to the patient or to a facility where the patient is under care. ‘Institutional Patient’ refers to a patient under care at a hospital, hospice, skilled-nursing facility, assisted-living facility, care home, home health and the like. In the same context, Healthcare Providers refers to a medical doctor (MD, DO), pharmacist, registered nurse practitioner (RN), physician assistant (PA), licensed vocational nurse (LVN) and the like.

In the present system of healthcare and pharmacy data management and administration method and system that may facilitate ordering, dispensing, reporting, billing, planning, outcome collection, outcome analysis and prediction, third-party data and services integration, real-time data collection and transfer, user interfaces, documentation, and the like related to a prescription, such as a prescribed medication, and to various patient-related events and outcomes. Various facilities may be included in a medical prescription validation and selection facility (alternatively referred to as a medication management facility), a dispensing and marking facility, a delivery facility, a prescription administration facility (also referred to herein as a medication administration facility), a patient attributes and events facility, an outcome events facility, an Enhanced Electronic Health Record (E-EHR)/an Enhanced Electronic Medical Record (E-EMR) facility, an outcome facility, an existing medical record facility, an outcome database facility, an outcome analytics facility, a rehabilitation and Long-term Care (“LTC”) facility. Each facility may individually be communicatively connected together through a network, such as the Internet, a proprietary network, a virtual private network, or the like, to facilitate real-time data capture and communication of data among the facilities.

The prescribing facility may allow a doctor, physician, nurse, pharmacist, or a healthcare specialist/professional to enter an order for a prescription of a medication or other medical care items for a patient. This may apply a plurality of identification methods to uniquely identify, record, and document details of a person entering the orders. The prescribing facility may be communicatively coupled in real-time to a backbone network. The backbone network may be a network that may be communicatively coupled to other facilities elsewhere and may carry medical data being communicated among the facilities, such as real-time medical data event packets, files, or streams. The exemplary medical event data being carried over the backbone may include information from any of the facilities including, without limitation, prescribing units, care planning facilities, laboratories and imaging centres, insurance and payments units, rehabilitation and LTC facilities, electronic medical record repositories, dashboards and databases. In an exemplary case, the backbone network may be the Internet, an intranet, a Wide Area Network, a Local Area Network, a wireless network, a personal area network, a virtual private network, or any other type of wired, wireless, or logical network including ad hoc, peer-to-peer, hub-and-spoke, tree, ring, serial, infrastructure-type protocols, and the like. The backbone network may also facilitate secure communication, using firewalls, among different facilities of the password protection, anti-virus protection, packet inspection, spam filtering, and other security facilities.

Further, the collaboration facility may obtain patient information via the backbone network. The information may include medical history, clinical outcomes, and allergic reactions to certain types of drugs and similar kind of information related to the patient. The prescribing facility may also allow the healthcare specialist to prescribe medication, such as through an electronic order entry facility. In an example, the electronic order entry facility may comprise an interface for aiding a healthcare specialist to prescribe the medication or treatment for the patient. The prescribing facility may present the obtained patient information to the healthcare specialist to help facilitate prescribing appropriate medication or treatment for the patient. Once the healthcare specialist enters the prescription for the patient in the electronic order entry facility, the prescribing facility may communicate the prescription to the backbone network or may send it directly to the prescription management facility, either already assigned to the patient or selected from a list of available options based on geolocation, hours of service and/or other selection/preference criteria necessary for such selection, and in embodiments directed to the prescription validation/selection facility therein. This communication may be done in real-time as an event. Further, the prescription may be made available to all other facilities via the backbone network. Care provider, at any time change, either permanently or temporarily, the prescription management facility for a given patient and may compile and transmit prescription data for the newly assigned prescription management facility to review and assess regulatory/patient compliance, safety, appropriateness of the prescribed regimen.

In an embodiment of the invention, the prescription from the collaboration facility may be raw prescription data associated with the patient. The prescription facility may include a computer that may transform the raw prescription data into a format such that it may be integrated with the patient's EHR in the E-EHR/E-EMR facility.

The prescription may be received by a doctor's validation and selection facility either directly from the prescribing facility or over the backbone network to which the validation and selection facility may be communicatively coupled. The validation and selection facility may be configured to receive/send real-time event tracking data with other patient medical-related facilities. Further, the validation and selection facility may also obtain information regarding the patient, including the past medical history of the patient, treatment outcomes for the patient (from past or current treatments), and allergic reactions to certain types of drugs/foods and similar kinds of information. The validation and selection facility may thus embody various features to ensure improved probability of selecting and delivering the appropriate medication to the patient, such as modules for identifying patient allergies or sensitivities, modules for identifying potential interactions among medications prescribed or proposed to be prescribed, and modules that use information or insights derived from the outcome module, such as to propose medications that have produced favorable and safe outcomes in other similar patients.

In an exemplary case, the validation and selection facility may be a pharmacy facility and may include the services of a pharmacist to validate the medical prescription of the patient. In another exemplary case, the pharmacist may be provided with a graphical user interface for performing validation of the medical prescription. Further, in such an interface, a user may utilize various buttons, list boxes, and text boxes for viewing, entering and managing data, such as patient data, medication data, outcome data, and order entry data. In order to confirm the validity of data entered, the software underlying the graphical user interface may validate the entered data, such as for proper format and for the absence of various problems, such as unfavorable interactions with other medications, conflict with patient allergy profile, indication of a likely unfavorable outcome, deviation for standards (such as in units prescribed, frequency of prescription, or the like).

By way of example, once the validation and selection facility has received the medical prescription and the other details and information about the patient, the validation and selection facility may verify formulary compliance, determine drug allergy information associated with the prescribed drugs, and confirm the drug interaction information associated with the prescribed medication. The validation and selection facility may issue alerts or warnings to the pharmacist, the healthcare specialist entering the medication order on a patient-by-patient basis to provide an opportunity for the prescriber to re-consider the prescription, in light of these warnings, and potentially make adjustments in the prescription before the validation and selection facility accepts the order into its processing facilities. After these activities have been accomplished and after reviewing all the relevant patient information, a pharmacist associated with the validation and selection facility may validate the patient medical prescription. This pharmacist-based validation may be done as a manual confirmation that follows a series of automated validation steps as described herein. The validation and selection facility may communicate the validated prescription of the patient to other facilities via the backbone network.

In an embodiment of the invention, the validated prescription from the validation and selection facility may be raw validated prescription data associated with the patient. The raw validated prescription data may not be in a format that may be integrated. The validation and selection facility may include a computer that may transform the raw validated prescription data to a format such that it may be integrated with the patient's facility.

The drug management facility may perform similar functions as those described above, with the alternative or additional functionality of being able to provide healthcare facility professionals with the information necessary to dispense medication. The prescription management facility may be in electronic communication with the medication supply interface and may provide notification which may be output through the medication supply interface that the medication is approved for administration and whether the medication is in the medication storage device. The medication management facility may be kept constantly up to date with regard to the inventory of the medication storage facility by the ordering and administration functions described herein.

The server may provide an interface to other devices including, without limitation, clients, other servers, printers, database servers, print servers, file servers, communication servers, distributed servers and the like. Additionally, this coupling and/or the connection may facilitate remote execution of program across the network. The networking of some or all of these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The system as per its further embodiments allows configuring Delivery options for instance including but not limited to time or date. It also provides facility specific or other related indicators selection for the delivery of medicines to the required location.

The system as per its additional embodiments facilitates the process and improves on signup process. When creating a new account, it would be great to send the invitation to new user (i.e. new hospice, new driver and new nurses). Upon signup through that invitation, an email or text will be sent to hospice, drivers and nurses with the link of portal and/or app with login info.

The system also facilitates addition of multiple pharmacy locations. The option of adding multiple pharmacies where each patient can be assigned to 1 or more pharmacies on priority basis and 1 which is close or convenient to the patient is assigned. Another pharmacy as a back-up and/or off-hour pharmacy can also be added.

When patient's pharmacy is switched, all orders placed/delivered/pending (with an option to select date range or 30 days by default) are automatically (or manually) complied into a report (Recent Rx Activity) and provider can send to new pharmacy.

The system also present Formulary and Non-Formulary designation. This can be defined as formulary for each hospice where each hospice will have a formulary i.e. list of medicine which will be covered. Anything not in the formulary will be marked as non-formulary. At the time of ordering nurse will know if the medicine is formulary or non-formulary.

The system also allows to add hospice which will need pharmacy assistance to upload their staff including doctors, nurses and patients. A role for pharmacy who can login to any required hospice and help them getting started or later offer some kind of help.

If the Subject Methods and Systems are embodied in a software program, the software program may be associated with a client that may include a file client, print client, domain client, internet client, intranet client and other variants such as secondary client, host client, distributed client and the like. The client may include one or more of memories, processors, computer-readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A pharmaceutical care management system to create, document, transmit, track, a pharmaceutical care order, the system comprising: a plurality of service application instances, devices under use by a plurality of healthcare provider, receive electronic medication orders, the medication management facility configured to perform at least one of:
a) A verification of formulary compliance:
The system facilitates the user through the input data for analysis and development of information flow.
A method and system for communication between health care providers and logistic personals for quick clear efficient and error-free real-time communication with feedback and follow up capabilities in the provision, assessment, tracking, documenting care-decisions in a hierarchical manner.
A determination of drug interaction information associated with the electronic medication orders;
A medication validation and billing facility in electronic communication with the medication management facility, the medication validation and billing facility configured to receive the electronic medication orders, provide information generating an order for pharmaceutical-care, seeking approval or opinion of or sharing such order with other care provider(s), transmitting such order to a single or plurality of information regarding formulary compliance, the drug allergy information, and the drug interaction information, and further comprising an interface for receiving input from a pharmacist to approve the electronic medication orders.
A method of assessing a list of prescription management facilities and assigning one or more such facilities to a given patient and changing assigned facility and transmitting previous prescription data to newly assigned facility.
A method of providing the care provider the options for the fulfilment of a treatment order and limiting the options, where necessary for a variety of logistical, contractual and/or preferential reasons.
A method of providing patients services from pharmacy on regular basis and backup basis;
A method of assigning an access for the care provider to a wide array or limited number of prescription choices, driven by the economical, contractual and/or preferential reasons.
A method of assigning delivery options based on multiple indicators including date, time and courier options.
A method of transmitting a treatment order to a third party which, in turn, is capable of transmitting the order to facilities outside the system of this invention and whereby such order can be fulfilled in accordance with regulatory compliance.
A method of making available to the care provider the status of the order whether at discrete stages of order fulfillment or real-time, automatic updates.
A method of assisting the care provider to select a treatment option either from within or outside of an approved formulary and alerting the care provider about cost differentials.
A method of configuring the system to define an autonomous or approval-requiring access level to service types whereby:
The approval-seeking step can be configured as per preferences of the care providing facility and/or licensing-privileges of the care provider.
A method of defining hierarchical based the access level to the prescription management facility.
A method inviting users and granting access to the system though secure communication and validating identity of the invited/intended users before access is granted.
A method of compiling, generating, exporting, printing, electronically transmitting treatment history reports with all details in accordance with regulatory requirements or any other business, professional, safety or analytical basis.
A method of providing tutorials and information regarding efficient use of the system.
